(19) United States
(12) Patent Application Publication
(10) Pub. No.: US 2025/0345391 A1
(43) Pub. Date: Nov. 13, 2025

(54) COMPOSITIONS AND METHODS FOR THE TREATMENT OF SEPSIS

(71) Applicant: Children's Hospital Medical Center, Cincinnati, OH (US)

(72) Inventor: Basilia Zingarelli, Cincinnati, OH (US)

(21) Appl. No.: 18/869,063

(22) PCT Filed: Jun. 2, 2023
(86) PCT No.: PCT/US23/24265

§ 371 Date: Nov. 25, 2024

Related U.S. Application Data
(60) Provisional application No. 63/348,553, filed on Jun. 3, 2022.

(51) Int. Cl. A61K 38/17; A61K 45/06; A61P 31/04; G01N 33/68

(52) U.S. Cl. CPC A61K 38/1709; A61K 45/06; A61P 31/04; G01N 33/68

(57) ABSTRACT

Disclosed are methods of treating sepsis in an individual in need thereof via administration of a therapeutically effective amount of a humanin protein, or an analog thereof, to the individual. In one aspect, the methods may comprise administration of the humanin analog colivelin for reducing lung, liver and kidney injury and systemic inflammation after an infection.

### CROSS-REFERENCE TO RELATED APPLICATIONS

This application claims priority to and benefit of U.S. Provisional Application Ser. No. 63/348,553, filed Jun. 3, 2022, the contents of which are incorporated in their entirety for all purposes.

### STATEMENT REGARDING FEDERALLY-SPONSORED RESEARCH

This invention was made with government support under GM115973 and GM067202 awarded by the National Institutes of Health. The government has certain rights in the invention.

### BACKGROUND

Sepsis is a life-threating organ dysfunction caused by dysregulated host responses to infection. A recent global study reported 49 million cases and 11 million sepsis-related deaths in 2017, accounting for approximately 20% of all annual deaths globally. Endothelial injury is a hallmark of systemic inflammatory response syndrome during sepsis and largely contributes to the serious clinical consequences of the infection such as increased vascular permeability, tissue edema, augmented adhesion of leukocytes and platelet aggregation, and loss of flow-dependent vasodilation leading to profound decrease in systemic vascular tone, and collapse of the microcirculation, and contributing to acute lung, kidney and liver injury. Improved treatments for sepsis, and endothelia injury in sepsis is needed. The instant disclosure seeks to address, but is not limited to, one or more of the aforementioned needs in the art.

### BRIEF SUMMARY

Disclosed are methods of treating sepsis in an individual in need thereof via administration of a therapeutically effective amount of a humanin protein, or an analog thereof, to the individual. In one aspect, the methods may comprise administration of the humanin analog colivelin for reducing lung, liver and kidney injury and systemic inflammation after an infection.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. Plasma levels of Syndecan-1 (A), Endoglin (B), ICAM-1 (C), P-selectin (D), PCSK9 (E), and Angiopoietin-2 (F) at 0 h, 6 h and 18 h after cecal ligation and puncture (CLP). Data represents the mean±SEM of 10-13 mice for group (n=10 control group at 0 h, n=13 at 6 h, n=11 at 18 h). *Represents P<0.05 versus control mice at time 0.

FIG. 2. Representative histology photomicrographs of lung sections of a control mouse (A) or mice subjected to polymicrobial sepsis at 6 h (B) and 18 h (C) after cecal ligation and puncture (CLP). Lung damage at 6 h and 18 h after CLP was characterized by severe reduction of alveolar space, neutrophil adhesion along vascular wall, hemorrhage, and infiltration of inflammatory cells. Magnification ×100. A similar pattern was seen in tissue sections of n=5 mice in each experimental group. (D) Histopathologic scores of lung sections (n=5 mice for each group). Lung injury was scored from 0 (no damage) to 16 (maximum damage). Box plots represent 25th percentile, median, and 75th percentile; error bars define 10th and 90th percentiles. *Represents P<0.05 versus control mice at time 0.

FIG. 3. Activity of myeloperoxidase (MPO) in lung (A), liver (B), kidney (C) at 6 h after cecal ligation and puncture (CLP). Data represents the mean±SEM of 7-17 mice for group (n=17 control group, n=13 vehicle group, n=7 colivelin 100 μg group, n=11 colivelin 200 μg group). *Represents P<0.05 versus control mice; #represents P<0.05 versus vehicle-treated mice. (D-E) Representative histology photomicrographs of lung sections of colivelin-treated mice at 6 h after CLP. Vehicle (200 μl distilled water) or colivelin (100 or 200 μg/kg) was administered intraperitoneally at 1 h after CLP. Magnification ×100. A similar pattern was seen in tissue sections of n=5 mice in each experimental group.

FIG. 4. Plasma levels of TNFα (A), MIP-1α (B), IL-10 (C), KC (D), IL-1β (E), and IL-6 (F) at 6 h after cecal ligation and puncture (CLP). Vehicle (200 μl distilled water) or colivelin (100 or 200 μg/kg) was administered intraperitoneally at 1 h after CLP. Data represents the mean±SEM of 4-7 mice for group (n=4 control group, n=6 vehicle group, n=4 colivelin 100 μg group, n=7 colivelin 200 μg group). *Represents P<0.05 versus control mice; #represents P<0.05 versus vehicle-treated mice.

FIG. 5. Plasma levels of Syndecan-1 at 6 h after cecal ligation and puncture (CLP). Vehicle (200 μl distilled water) or colivelin (100 or 200 μg/kg) was administered intraperitoneally at 1 h after CLP. Data represents the mean±SEM of 7-13 mice for group (n=10 control group, n=13 vehicle group, n=7 colivelin 100 μg group, n=11 colivelin 200 μg group). *Represents P<0.05 versus control mice; #represents P<0.05 versus vehicle-treated mice.

FIG. 6. Transmission electron microscopy sections of thoracic aortas with lanthanum staining at 6 h after cecal ligation and puncture (CLP). Panels of control mice (A, D, G, J) show thick endothelial glycocalyx layer with dense individual bundles and normal mitochondria in endothelial and smooth muscle cells. Panels of vehicle-treated mice (B, E, H, K) show thin glycocalyx with bundles with loose structure and some damaged mitochondria and authophagic vesicles at 6 h after CLP. Panels of colivelin-treated mice (C, F, I, L) show well preserved thin glycocalyx and normal mitochondria at 6 h after CLP. Vehicle (200 μl distilled water) or colivelin (100 μg/kg) was administered intraperitoneally at 1 h after CLP. Arrows=glycocalyx; EC=endothelial cell; EL=elastica lamina; EM=extracellular matrix; RC=red cell; SMC=smooth muscle cell; a=authophagosome; n=nucleus; m=normal mitochondria; dm=damaged swollen mitochondria presenting translucent matrix and disrupted cristae.

FIG. 7. Representative Western blots of total STAT3, p-STAT3(Ser727) and p-STAT3(Tyr705) in cytosol and nuclear extracts of thoracic aorta; β-actin was used as loading control protein (A). Image analyses of cytosol and nuclear of relative intensity of total STAT3 (B), ratio of p-STAT3(Ser727)/STAT3 (C), and ratio of p-STAT3(Tyr705)/STAT3 (D) as determined by densitometry. Vehicle (200 μl distilled water) or colivelin (100 μg/kg) was administered intraperitoneally at 1 h after CLP. Each data represents the mean±SEM of 3-4 animals for each group. *Represents P<0.05 versus control mice; #represents P<0.05 versus vehicle-treated mice.

FIG. 8. Representative Western blots of total STAT3, p-STAT3(Ser727) and p-STAT3(Tyr705) in lung cytosol and nuclear extracts; β-actin was used as loading control protein (A). Image analyses of cytosol and nuclear of relative intensity of total STAT3 (B), ratio of p-STAT3(Ser727)/STAT3 (C), and ratio of p-STAT3(Tyr705)/STAT3 (D) as determined by densitometry. Vehicle (200 μl distilled water) or colivelin (100 μg/kg) was administered intraperitoneally at 1 h after CLP. Each data represents the mean±SEM of 3-4 mice for group (n=3 control group, n=4 vehicle group, n=4 colivelin 100 Vg group). *Represents P<0.05 versus control mice; #represents P<0.05 versus vehicle-treated mice.

FIG. 9. Representative Western blots of total AMPKα and pAMPKα in cytosol extracts of thoracic aorta; β-actin and Revert stain were used to verify loading of proteins (A). Image analyses of ratio of relative intensity of p-AMPKα/AMPKα (B) as determined by densitometry. Vehicle (200 μl distilled water) or colivelin (100 μg/kg) was administered intraperitoneally at 1 h after CLP. Each data represents the mean±SEM of 3-4 mice for group (n=3 control group, n=4 vehicle group, n=4 colivelin 100 μg group). *Represents P<0.05 versus control mice; #represents P<0.05 versus vehicle-treated mice.

FIG. 10. Survival rate (A), severity score (B) and body weight loss (C) of mice at 7 days after cecal ligation and puncture (CLP). Mice were subjected to CLP and received colivelin (100 μg/kg subcutaneously) or vehicle at 1 h, 3 h and 24 h after CLP. All mice received fluid resuscitation (35 ml/kg normal saline with 5% dextrose subcutaneously) every 24 h up to 7 days and ceftriaxone (25 mg/kg) and metronidazole (12.5 mg/kg) intraperitoneally every 12 h up to 3 days after the CLP procedure. *Represents P<0.05 versus colivelin-treated mice.

FIG. 11. Representative histology photomicrographs of thoracic aorta sections of a control mouse (A) or mice subjected to polymicrobial sepsis at 6 h (B) and 18 h (C) after cecal ligation and puncture (CLP) with normal morphology of tunica intima (TI), tunica media (TM), and tunica adventitia (TA). Magnification ×400. A similar pattern was seen in n=4-8 different tissue sections in each experimental group.

FIG. 12. Plasma levels of Syndecan-1 (A), Endoglin (B), ICAM-1 (C), P-selectin (D), PCSK9 (E), and Angiopoietin-2 (F) at 0 h, 6 h and 18 h after cecal ligation and puncture (CLP) or sham surgery (SHAM). Data represents the mean±SEM of 10-13 mice for group (n=10 control group at 0 h, n=13 at 6 h CLP, n=11 at 18 h CLP, n=9 at 6 h SHAM, n=8 at 18 h SHAM). *Represents P<0.05 versus control mice at time 0; #represents P<0.05 versus sham mice.

FIG. 13. Activity of myeloperoxidase (MPO) in lung (A), liver (B), kidney (C) at 6 h after cecal ligation and puncture (CLP) or sham surgery (SHAM). Data represents the mean±SEM of 8-17 mice for group (n=17 control group at 0 h, n=13 at 6 h CLP, n=11 at 18 h CLP, n=9 at 6 h SHAM, n=8 at 18 h SHAM). *Represents P<0.05 versus control mice at time 0; #represents P<0.05 versus sham mice

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting. The methods may comprise, consist of, or consist essentially of the elements of the compositions and/or methods as described herein, as well as any additional or optional element described herein or otherwise useful in the treatment of sepsis in an individual in need thereof.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “therapeutically effective amount” or “effective amount” mean the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

The peptides and methods hereof may also comprise administering pro-drugs that metabolize to an active form of these peptides. As used herein, a “pro-drug” is a compound that a biological system metabolizes to an active compound as a result of spontaneous chemical reaction(s), enzyme catalyzed reaction(s), and/or metabolic chemical reaction(s), or a combination of each. Exemplary prodrugs may be formed using groups attached to functionality, e.g. HO—, HS—, HOOC—, R2N-, associated with the drug, that cleave in vivo. Further exemplary prodrugs include, but are not limited to, carboxylate esters where the group is alkyl, aryl, aralkyl, acyloxyalkyl, alkoxycarbonyloxyalkyl as well as esters of hydroxyl, thiol and amines, where the group attached is an acyl group, an alkoxycarbonyl, aminocarbonyl, phosphate or sulfate. The groups illustrated are exemplary and not exhaustive, and the present disclosure includes other known varieties of prodrugs.

“Sequence identity” as used herein indicates a nucleic acid or peptide sequence that has the same sequence as a reference sequence or has a specified percentage of nucleotides or amino acids that are the same at the corresponding location within a reference sequence when the two sequences are optimally aligned. For example a nucleic acid or peptide sequence may have at least 50%, 60%, 70%, 75%, 80%, 85%, 90%, 95%, 96%, 97%, 98%, 99%, or 100% identity to the reference nucleic acid or peptide sequence. The length of comparison sequences will generally be at least 5 contiguous nucleotides, or amino acids, or at least 10, 11, 12, 13, 14, 15, 16, 17, 18, 19, 20, 21, 22, 23, 24, 25 or more contiguous nucleotides or amino acids.

Disclosed herein are methods of treating sepsis in an individual in need thereof. In one aspect, the methods may comprise administering a therapeutically effective amount of a humanin protein, or an analog thereof, to said individual. In one aspect, the sepsis may be accompanied by one or both of sepsis-associated endothelial dysfunction and organ injury. In one aspect, humanin or a humanin analog may be used in the treatment of organ injury consequent to sepsis. For example, the methods may comprise administration of humanin-G to an individual in need thereof to reduce lung injury after polymicrobial peritonitis. In a further aspect, the methods may comprise administration of colivelin to an individual in need thereof reduces lung, liver and kidney injury and systemic inflammation after polymicrobial peritonitis.

In one aspect, the humanin protein or analog thereof may comprise a peptide having from about 75% sequence identity, or about 80% sequence identity, or about 85% sequence identity, or about 90% sequence identity, or about 95% sequence identity to a sequence of Table 1. In one aspect, the humanin protein or analog thereof may have at least 90%, or at least 95% sequence homology to a peptide having a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In one aspect, the humanin protein or analog thereof may comprise a peptide having from about 75% sequence identity, or about 80% sequence identity, or about 85% sequence identity, or about 90% sequence identity, or about 95% sequence identity to a sequence of Table 1, wherein said protein or analog thereof comprises a sequence selected from CLLLTSEIDLP (SEQ ID NO: 9), LLLLT (SEQ ID NO: 10), EIDLP (SEQ ID NO: 11), or combinations thereof. In one aspect, the humanin protein or analog thereof may be selected from HN, HNG, HNA, AGA-HNG, HN17, HNG17, AGA-C8R-HNG17, colivelin, and combinations thereof.

TABLE 1
Humanin derivatives and colivelin (Source: Matsuoka M. et al, Humanin
and Colivelin: Neuronal-Death-Suppressing Peptides for Alzheimer's
Disease and Amyotrophic Lateral Sclerosis, CNS Drug Reviews, Vol. 12,
No. 2, pp. 113-122 (2006).)

| Name | Sequence | SEQ ID NO |
|---|---|---|
| Humanin (HN) | MAPRGFSCLLLTSEIDLPVKRRA | SEQ ID NO: 1 |
| Humanin G (HNG) | MAPRGFSCLLLLTGEIDLPVKRRA | SEQ ID NO: 2 |
| HNA | MAPRGFSALLLLTSEIDLPVKRRA | SEQ ID NO: 3 |
| AGA-HNG | MAAGAFSCLLLLTSEIDLPVKRRA | SEQ ID NO: 4 |
| HN17 | PRGFSCLLLLTSEIDLP | SEQ ID NO: 5 |
| HNG17 | PRGFSCLLLLTGEIDLP | SEQ ID NO: 6 |
| AGA-C84-HNG17 | PAGASRLLLLTGEIDLP | SEQ ID NO: 7 |
| Colivelin | SALLRSIPAPAGASRLLLLTGEIDLP | SEQ ID NO: 8 |

In one aspect, the humanin protein or analog thereof may be colivelin, having the sequence SALLRSIPAPAGASRLLLLTGEIDLP (SEQ ID NO: 8).

In one aspect, the humanin protein or analog thereof may be modified. In one aspect, the modification may be that the humanin protein or analog thereof is PEGylated. In other aspects, the humanin protein or analog thereof may be administered as a pro-drug. In certain aspects, the humanin protein or analog thereof may be produced synthetically, and may, for example, comprise modified amino acids and/or D-amino acids which correspond to the L-amino acids of the listed sequences.

The humanin protein or analog thereof may be administered in an amount and duration as determined by the treating physician. For example, the amount and duration may be sufficient to obtain an improvement of lung injury, reduced leukosequestration in lung, liver and kidney, a reduction of circulating syndecan-1, and combinations thereof. In one aspect, the amount and duration may be sufficient to reduce one or both of MPO activity or neutrophil infiltration in one or more of lungs, liver, and kidney. In a further aspect, the amount and duration may be sufficient to reduce levels of TNF-α, MIP-1α and IL-10 in the individual. In one aspect, the amount and duration may be sufficient to reduce cytosolic or nuclear expression of both pSTAT3(Ser727) and pSTAT3(Tyr705) in the individual. In a further aspect, the amount and duration sufficient to reduce systemic elevation of TNFα, MIP-1α, KC and IL-10 in said individual.

Administration of the humanin protein or analog thereof may take a variety of forms. For example, the humanin protein or analog thereof may be administered via intramuscular (IM) delivery, intravenous (IV) delivery, subcutaneous (SC) delivery, intra-arterial delivery, oral delivery, gavage delivery, emollient/skin delivery, transdermal patch, and/or intranasally. The humanin protein or analog thereof may be administered continuously or as a dose. Exemplary dosing includes administering for at a period of at least one hour, or at least one day, or at least two days, or at least three days, or at least four days, or at least five days, or at least six days, or at least one week. The humanin protein or analog thereof may be administered within two hours of admission or diagnosis, within three hours of admission or diagnosis, within four hours of admission or diagnosis, within five hours of admission or diagnosis, or within six hours of admission or diagnosis.

Administration of the humanin protein or analog thereof may include administration of an amount of from about 50 μg/kg to 1000 μg/kg, from about 100 μg/kg to about 800 μg/kg, from about 200 μg/kg to about 600 μg/kg, or from about 300 μg/kg to about 500 μg/kg.

The sepsis being treated via the disclosed methods may be due to a variety of bacteria types, including but not limited to Staphylococcus aureus, Pseudomonas aeuroginosa, Klebsiella species, Streptococcus species, Salmonella species, Shigella species, Mycobacterium tuberculosis, Enterococcus species, Enterobacteriaceae, E coli, Clostridium species, Neisseria gonnorrhoea, Acinetoebacter baumannii, Campylobacter species, and combinations thereof.

In one aspect, the humanin protein or analog thereof may be administered before, after, or simultaneously with a therapeutic amount of one or both of an antibiotic and a fluid. In certain aspects, the humanin protein or analog thereof may be added to a fluid or antibiotic composition which is then administered to the individual in need thereof. The antibiotic may be a broad-spectrum antibiotic, for example, an antibiotic selected from an aminoglycoside, ampicillin, amoxicillin, clavulanic acid (Augmentin), a carbapenem (e.g. imipenem), piperacillin, tazobactam, a quinolone (e.g. ciprofloxacin), a tetracycline, chloramphenicol, ticarcillin, trimethoprim, sulfamethoxazole (Bacterium), and combinations thereof, more specifically an antibiotic selected from methicillin, vancomycin, linezolid, daptomycin, quinupristin, dalfopristin, teicoplanin, cephalosporin, carbapenem, fluoroquinolone, aminoglycoside, colistin, erythromycin, clindamycin, beta-lactam, macrolide, amoxicillin, azithromycin, penicillin, ceftriaxone, azithromycin, ciprofloxacin, isoniazid (INH), rifampicin (RMP), amikacin, kanamycin, capreomycin, trimethoprim, itrofurantoin, cefalexin, amoxicillin, metronidazole (MTZ), cefixime, tetracycline, meropenem, and combinations thereof. In other aspects the antibiotic may be a beta-lactam antibiotic, a broad-spectrum carbapenem, a fluoroquinolone, a macrolide, an aminoglycoside, and combinations thereof.

In a further aspect, a method of treating an individual for sepsis is disclosed, the method comprising detecting an elevated level of a biomarker selected from tumor necrosis factor-α (TNFα), interleukin (IL)-1β, IL-6, IL-10, keratinocytes-derived chemokine (KC), macrophage inflammatory proteins (MIP-1α), endoglin, PCSK9, ICAM-1, P-selectin, syndecan-1, and combinations thereof in said individual; and administering a therapeutically effective amount of a humanin protein, or analog thereof, to said individual, as described herein.

### Pharmaceutical Compositions

In one aspect, active agents provided herein may be administered in a dosage form selected from parenteral injection, continuous injection, oral administration, nasal administration, ophthalmic administration, buccal administration, and transdermal administration. In some aspects, the peptides provided herein may be formulated into liquid preparations for, e.g., oral administration.

In one aspect, the peptide-containing compositions may be isotonic with the blood or other body fluid of the recipient. The isotonicity of the compositions may be attained using, for example, sodium tartrate, propylene glycol or other inorganic or organic solutes such as sodium chloride. Buffering agents may be employed, such as acetic acid and salts, citric acid and salts, boric acid and salts, and phosphoric acid and salts. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

A pharmaceutically acceptable preservative may be employed to increase the shelf life of the pharmaceutical compositions, such as benzyl alcohol, parabens, thimerosal, chlorobutanol, or benzalkonium chloride. Preservatives may be added in an amount of from about 0.02% to about 2% based on the total weight of the composition, although larger or smaller amounts may be desirable depending upon the agent selected. Reducing agents, as described above, may be advantageously used to maintain good shelf life of the formulation.

In one aspect, the peptides provided herein may be in admixture with a suitable carrier, diluent, or excipient such as sterile water, physiological saline, glucose, or the like, and may contain auxiliary substances such as wetting or emulsifying agents, pH buffering agents, gelling or viscosity enhancing additives, preservatives, flavoring agents, colors, and the like, depending upon the route of administration and the preparation desired. Such preparations may include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components may influence the physical state, solubility, stability, rate of in vivo release, and rate of in vivo clearance, and thus may be chosen according to the intended application, such that the characteristics of the carrier are tailored to the selected route of administration.

For oral administration of a peptide, the pharmaceutical compositions may be provided via a lipid-based nanocarrier such as, for example, one or more of oil-in-water nanoemulsions, self-emulsifying drug delivery systems (SEDDS), solid lipid nanoparticles (SLN), nanostructured lipid carriers (NLC), or liposomes and micelles.

In some aspects, the amount of peptide to be delivered or contained within a unit dose may be from about 1 mg or less to about 1,000 mg or more of a active agent provided herein, for example, from about 10, 20, 30, 40, 50, 60, 70, 80, 90, or 100 mg to about 150, 200, 250, 300, 350, 400, 450, 500, 550, 600, 650, 700, 750, 800, or 900 mg. A dosage appropriate to the patient and the number of doses to be administered daily may be selected. In certain aspects, two or more therapeutic agents (such as a peptide and antibiotic) may be incorporated to be administered into a single dosage form (e.g., in a combination therapy); however, in other aspects, the therapeutic agents may be provided in separate dosage forms.

In one aspect, the peptide is administered via injection. The duration of the therapy may be adjusted depending upon various factors, and may comprise a single injection administered daily, or twice a day, or three times a day, or over a longer period of time, such as every other day, every two days, every three days, every four days, every five days, every six days, every seven days, or weekly, every two weeks, every three weeks, or monthly. In other aspects, the peptide may be administered via continuous intravenous administration.

In some aspects, the active agents provided herein may be provided to an administering physician or other health care professional in the form of a kit. The kit is a package which houses a container which contains the active agent(s) in a suitable pharmaceutical composition, and instructions for administering the pharmaceutical composition to a subject. The kit may optionally also contain one or more additional therapeutic agents currently employed for treating a disease state as described herein. For example, a kit containing one or more compositions comprising active agents provided herein in combination with one or more additional active agents may be provided, or separate pharmaceutical compositions containing an active agent as provided herein and additional therapeutic agents may be provided. The kit may also contain separate doses of a active agent provided herein for serial or sequential administration. The kit may optionally contain one or more diagnostic tools and instructions for use. The kit may contain suitable delivery devices, e.g., syringes, and the like, along with instructions for administering the active agent(s) and any other therapeutic agent. The kit may optionally contain instructions for storage, reconstitution (if applicable), and administration of any or all therapeutic agents included. The kits may include a plurality of containers reflecting the number of administrations to be given to a subject.

### Examples

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

Colivelin, a synthetic derivative of humanin, ameliorates endothelial injury and glycocalyx shedding after sepsis in mice

Endothelial dysfunction plays a central role in the pathogenesis of sepsis-mediated multiple organ failure. Several clinical and experimental studies have suggested that the glycocalyx is an early target of endothelial injury during an infection. Colivelin, a synthetic derivative of the mitochondrial peptide humanin, has displayed cytoprotective effects in oxidative conditions. Applicant aimed to determine the potential therapeutic effects of colivelin in endothelial dysfunction and outcomes of sepsis in vivo. Male C57BL/6 mice were subjected to a clinically relevant model of polymicrobial sepsis by cecal ligation and puncture (CLP) and were treated with vehicle or colivelin (100-200 μg/kg) intraperitoneally at 1 h after CLP. Vehicle-treated mice had early elevation of plasma levels of the adhesion molecules ICAM-1 and P-selectin, the angiogenetic factor endoglin and the glycocalyx syndecan-1 at 6 h after CLP when compared to control mice, while levels of angiopoietin-2, a mediator of microvascular disintegration, and the proprotein convertase subtilisin/kexin type 9, an enzyme implicated in clearance of endotoxins, raised at 18 h after CLP. The early elevation of these endothelial and glycocalyx damage biomarkers coincided with lung histological injury and neutrophil inflammation in lung, liver, and kidneys. At transmission electron microscopy analysis, thoracic aortas of septic mice showed increased glycocalyx breakdown and shedding, and damaged mitochondria in endothelial and smooth muscle cells. Treatment with colivelin ameliorated lung architecture, reduced organ neutrophil infiltration, and attenuated plasma levels of syndecan-1, tumor necrosis factor-α, macrophage inflammatory protein-1α and interleukin-10. These therapeutic effects of colivelin were associated with amelioration of glycocalyx density and mitochondrial structure in the aorta. At molecular analysis, colivelin treatment was associated with inhibition of the signal transducer and activator of transcription 3 and activation of the AMP-activated protein kinase in the aorta and lung. In long-term outcomes studies up to 7 days, co-treatment of colivelin with antimicrobial agents significantly reduced the disease severity score when compared to treatment with antibiotics alone. In conclusion, Applicant's data support that damage of the glycocalyx is an early pathogenetic event during sepsis and that colivelin may have therapeutic potential for the treatment of sepsis-associated endothelial dysfunction.

Clinical and experimental studies have proven that the glycocalyx is one of the earliest sites involved during the pathogenesis of endothelial injury. The glycocalyx is a gel-like mesh layer which covers the luminal surface of vascular endothelial cells. It is composed of membrane-attached proteoglycans, glycosaminoglycan sidechains, glycoproteins, and adherent plasma proteins such as albumin and antithrombin. This structure is known to play critical roles in maintaining hemostasis and coagulation, regulating leukocyte adhesion and rolling, and sensing mechanical forces, such as shear stress and pressure. It also shields cell surface receptors and can prevent their activation by presenting a physical barrier. In sepsis, there is a distinct alteration in the composition of the endothelial glycocalyx following the activation of proteases, such as metalloproteinases, heparanase, and hyaluronidase, by bacterial and inflammatory insults. These enzymes lead to glycocalyx degradation via release of glycosaminoglycan sidechains, and if severe enough, loss of core membrane proteins. As the glycocalyx is shed, circulating levels of glycocalyx components, including syndecans, can be measured and are considered biomarkers of endothelial injury.

Mitochondria have emerged as important players in maintaining vascular homeostasis. In addition to energy production, mitochondria affect a variety of complex processes including inflammation and cell survival. Mitochondria-derived peptides, including humanin, encoded by short open reading frame in the mitochondrial DNA (mtDNA), have been recently described to have biological effects. Several experimental studies describe potent cytoprotective effects of humanin and its synthetic derivatives. For example, humanin is shown to protect endothelial cells from oxidative stress and to prevent glucose-induce endothelial expression of adhesion molecules and apoptosis. At the molecular level, humanin appears to regulate metabolic homeostasis through involvement of the signal transducer and activator of transcription 3 (STAT3) and AMP-activated protein kinase (AMPK). Recently, colivelin, a new generation potent humanin derivative has also been reported to have cytoprotective effects by inhibiting apoptosis and inflammatory response in vitro and in vivo models of neuronal degeneration and ischemic injury (Chiba T et al. Development of a femtomolar-acting humanin derivative named colivelin by attaching activity-dependent neurotrophic factor to its n terminus: characterization of colivelin-mediated neuroprotection against alzheimer's disease-relevant insults in vitro and in vivo. J Neurosci (2005) 25:10252-61. doi: 10.1523/JNEUROSCI.3348-05.2005; Chiba T et al. Colivelin prolongs survival of an ALS model mouse. Biochem Biophys Res Commun (2006) 343:793-8. doi: 10.1016/j.bbrc.2006.02.184; Sari Y et al. A novel peptide, colivelin, prevents alcohol-induced apoptosis in fetal brain of C57BL/6 mice: signaling pathway investigations. Neuroscience (2009) 164:1653-64. doi:10.1016/j.neuroscience.2009.09.049; and Zhao H et al., Colivelin rescues ischemic neuron and axons involving JAK/STAT3 signaling pathway. Neuroscience (2019) 416:198-206. doi:10.1016/j. neuroscience.2019.07.020)). Despite the substantial literature on colivelin-mediated beneficial effects in neurological diseases, the effect of colivelin on the endothelial damage during a systemic inflammation, like sepsis, has not been investigated.

In the present study, by employing a clinically relevant mouse model of sepsis Applicant hypothesized that endothelial damage occurs early during sepsis and is characterized by structural damage of glycocalyx and associated with organ dysfunction and sought to evaluate the therapeutic efficacy of colivelin in sepsis and its potential molecular mechanisms of action.

### Results

Glycocalyx shedding and endothelial damage occur early during polymicrobial sepsis and are associated with lung injury

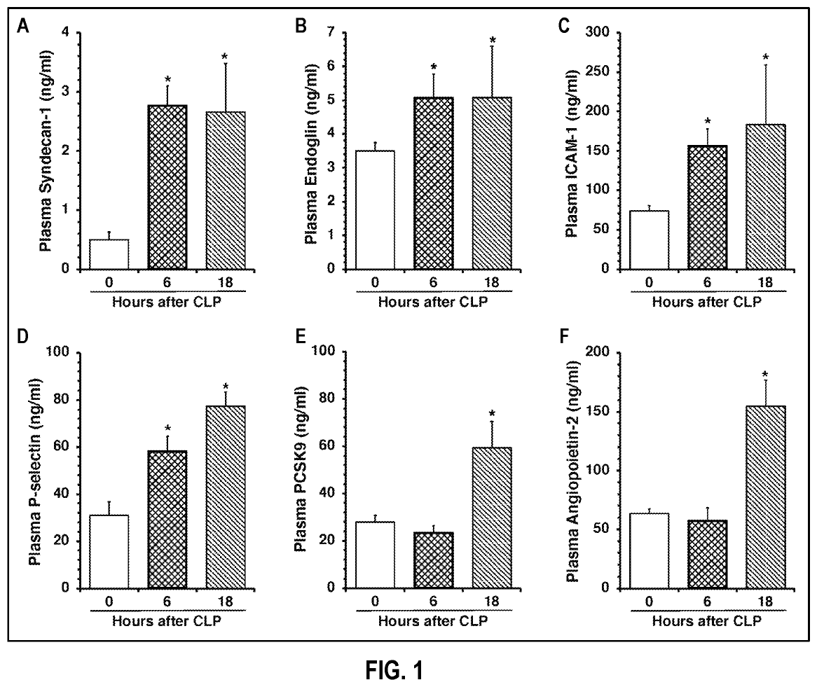

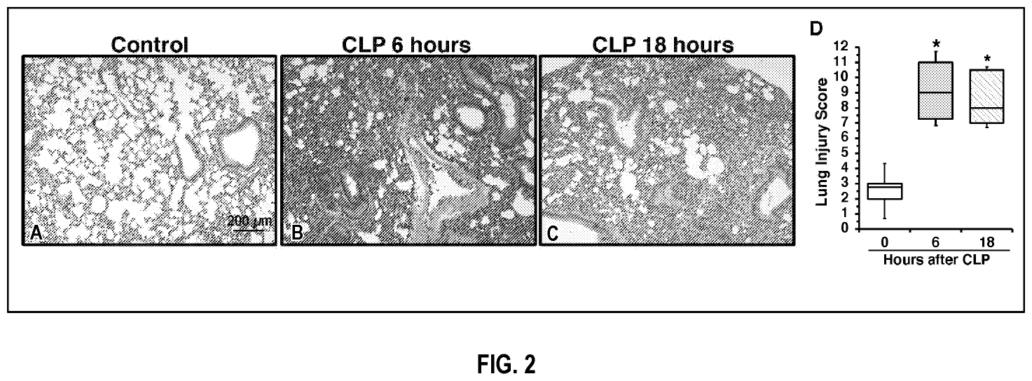

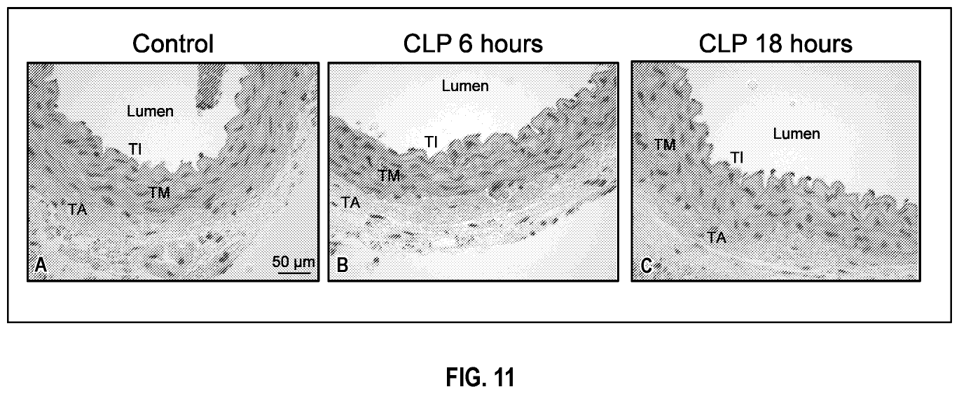

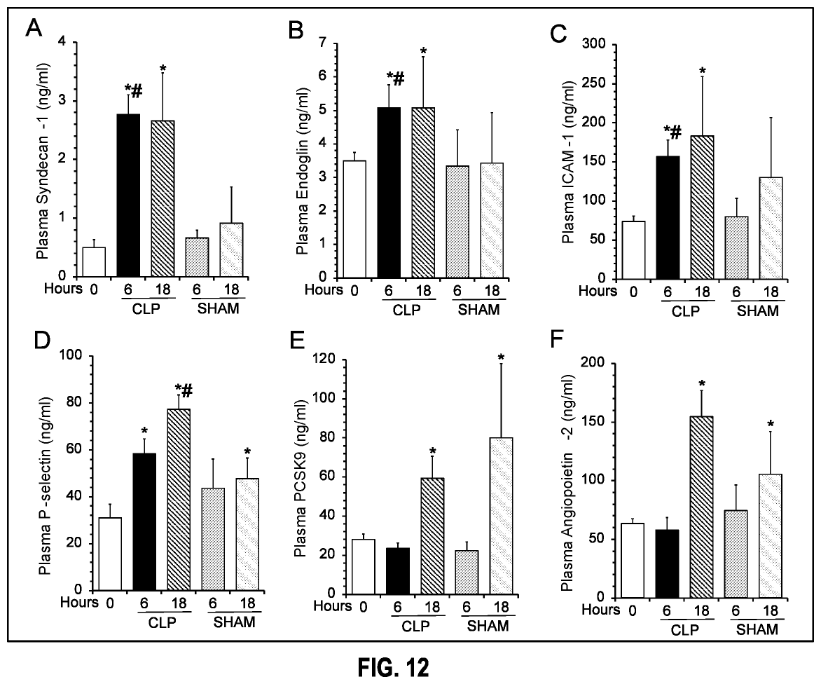

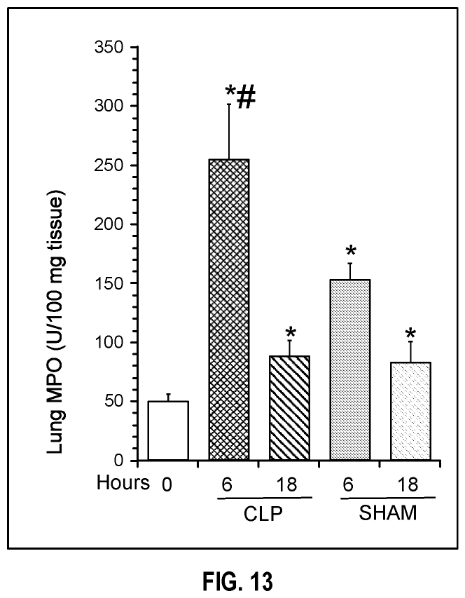

To determine the onset of endothelial damage, Applicant performed histology of thoracic aortas and we measured plasma biomarkers at 6 h and 18 h after CLP. Hematoxylin and eosin-stained sections of the thoracic aorta did not reveal alteration of cellular density or irregularities in the tunica intima, tunica media, and adventitia layers at 6 h or 18 h after CLP (FIG. 11). However, an early elevation of plasma levels of syndecan-1, a marker of glycocalyx breakdown and shedding, was observed at 6 h in mice subjected to CLP when compared to control mice at baseline conditions (2.77±0.34 versus 0.49±0.13 ng/ml, P<0.05; FIG. 1A). This early glycocalyx damage was also associated with an early increase of the angiogenetic factor endoglin (5.07±0.69 ng/ml), the adhesion molecules ICAM-1 (156.72±20.93 ng/ml) and P-selectin (58.26±6.40 ng/ml) when compared to control mice (3.50±0.24, 73.96±6.62, and 31.04±5.73 ng/ml, respectively; P<0.05). At 18 h after CLP plasma syndecan-1, endoglin, ICAM-1 and P-selectin were still maintained at high levels (FIGS. 1B-D). At 18 h after CLP, septic mice also exhibited higher plasma levels of angiopoietin-2 (154.80±21.90 ng/mL), a mediator of microvascular disintegration, and levels of PCSK9 (59.27±11.28 ng/mL), an enzyme implicated in low-density lipoprotein receptor degradation and clearance of endotoxins, when compared to control mice (63.19±4.08 and 27.99±2.80, respectively; P<0.05) (FIGS. 1E, F). Early degradation of endothelial glycocalyx was also associated with higher lung injury score at 6 h, which persisted at 18 h after CLP, and was characterized by reduced alveolar space, and accumulation of red and inflammatory cells when compared to control mice at basal condition (FIG. 2). To distinguish whether early endothelial damage was secondary to specific sepsis-induced immune response, we also measured these circulating biomarkers in sham mice, which underwent laparotomy but not CLP. In sham mice at 6 h, levels were not significantly different when compared with baseline levels of control mice. Sham mice at 18 h exhibited a significant elevation of P-selectin, PCSK9 and angiopoietin-2 (FIG. 12). There was only a mild infiltration of neutrophil, as determined by MPO activity, in the lung at 6 h when compared with control mice, but levels were significantly lower than mice subjected to CLP (FIG. 13). Thus, these data suggested that the early occurrence of endothelial damage is a specific sepsis-induced response and not induced by the sterile inflammation caused by the surgical procedures.

Treatment with Colivelin Reduces Neutrophil Infiltration in Lung, Liver and Kidney after CLP in a Dose-Independent Manner

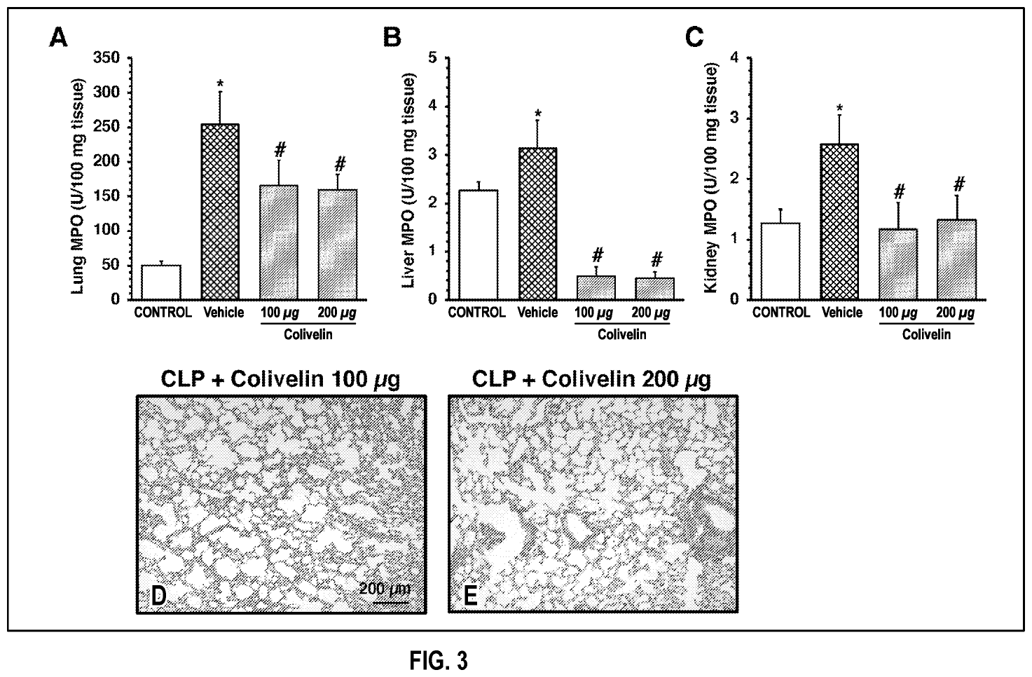

Considering the early elevation in plasma levels of adhesion molecules, Applicant next determined the effects of treatment with the peptide colivelin on neutrophil infiltration by measuring MPO activity in major organs at 6 h after CLP. Vehicle-treated mice had higher MPO activity in lungs, liver and kidneys when compared to control mice at basal conditions. Treatment with colivelin significantly decreased MPO activity in lungs, liver and kidneys in a dose-independent manner when compared to vehicle treatment (FIGS. 3A-C). Microscopic examination of hematoxylin and eosin-stained lung sections confirmed that treatment with colivelin reduced infiltration of inflammatory cells and ameliorated alveolar damage in the lung (FIGS. 3D, E) when compared to vehicle treatment (FIG. 2B).

Treatment with Colivelin Reduces Plasma Levels of Cytokines after CLP in a Dose-Independent Manner

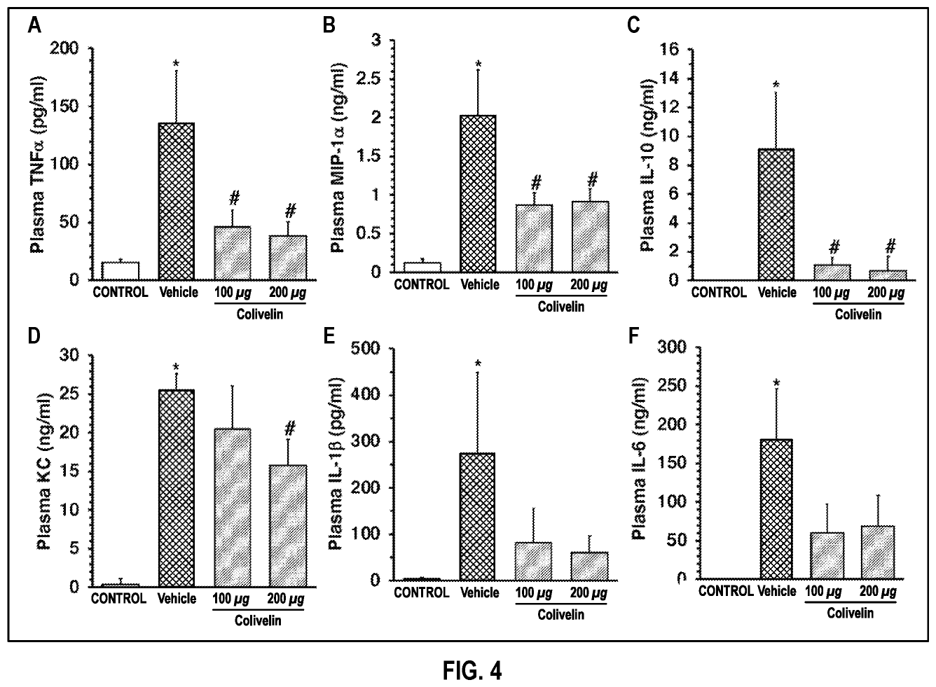

To evaluate the effect of colivelin on systemic inflammatory response, a panel of Th1/Th2/Th17 cytokines was measured. At 6 h after CLP, plasma levels of IL-1β, IL-6, IL-10, KC, TNF-α, and MIP-1α were significantly increased in vehicle-treated mice compared to control mice. Colivelin treatment significantly decreased levels of TNF-α, MIP-1α and IL-10 in a dose independent-manner. Levels of KC were significantly reduced in the mice treated with colivelin at 200 μg/kg. There was also a trend towards reduction of IL-1β and IL-6 after treatment with colivelin, but levels of these cytokines were not statistically different when compared with vehicle treatment (FIG. 4).

Treatment with Colivelin Ameliorates Endothelial Glycocalyx Damage and Mitochondrial Damage in Thoracic Aortas after CLP

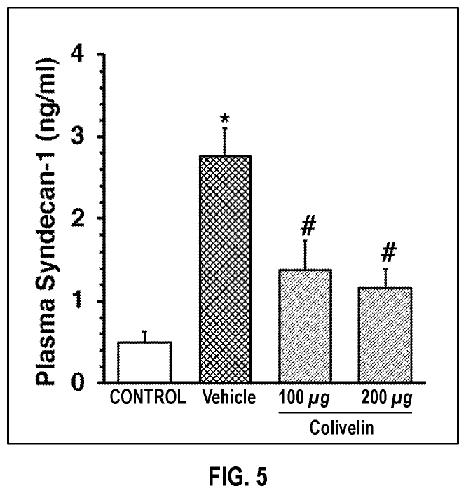

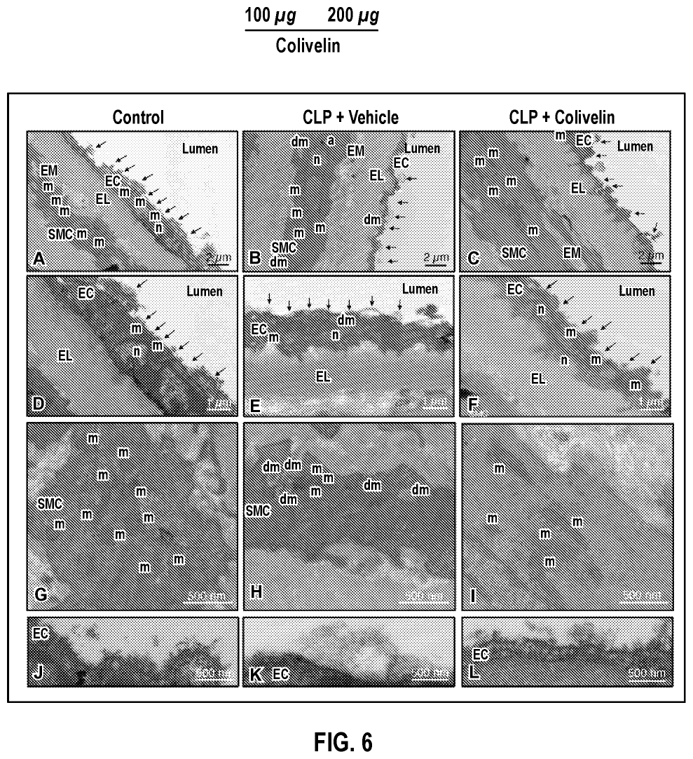

Applicant next evaluated the effect of colivelin on endothelial injury. Colivelin treatment significantly decreased levels of plasma syndecan-1 in a dose-independent manner at 6 h after CLP, thus suggesting reduction in glycocalyx shedding (FIG. 5). Since effects of the peptide were in a dose-independent manner, the ultrastructural changes of the thoracic aortas in mice treated with colivelin at 100 μg/kg only were examined (FIG. 6). At electron microscopic analysis, mitochondria damage was evident in smooth muscle and endothelial cells in vehicle-treated mice at 6 h after CLP and was characterized by swollen mitochondria and presence of autophagosomes when compared to control mice. On the luminal surface the lanthanum staining showed a thick endothelial glycocalyx layer with dense individual bundles in control mice. At 6 h after CLP, the glycocalyx layer appeared thinner with less dense bundles with loose structure in vehicle-treated mice. On the contrary, in colivelin-treated mice mitochondria appeared normal with dense matrix in all cell types and the dense structure of glycocalyx appeared well preserved when compared to vehicle treatment (FIG. 6).

Treatment with Colivelin Inhibits STAT3 Activation in Thoracic Aortas and Lungs after CLP

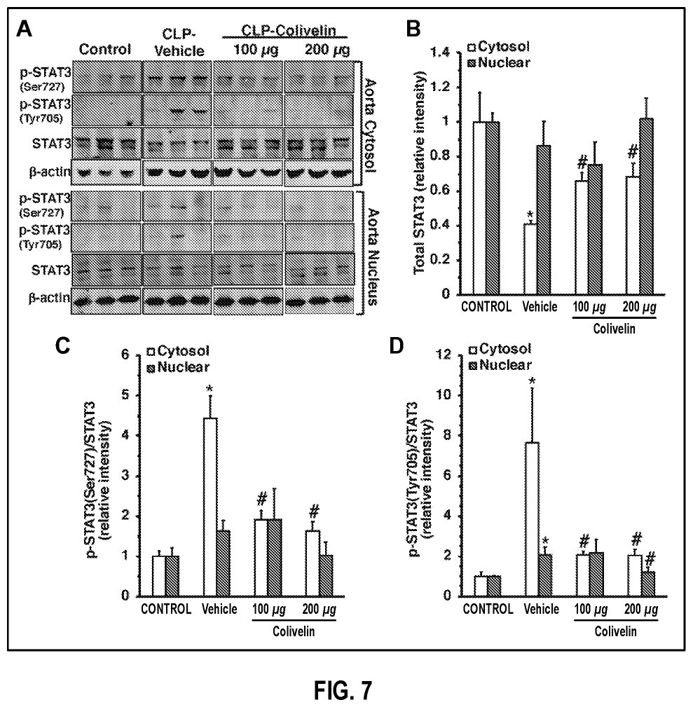

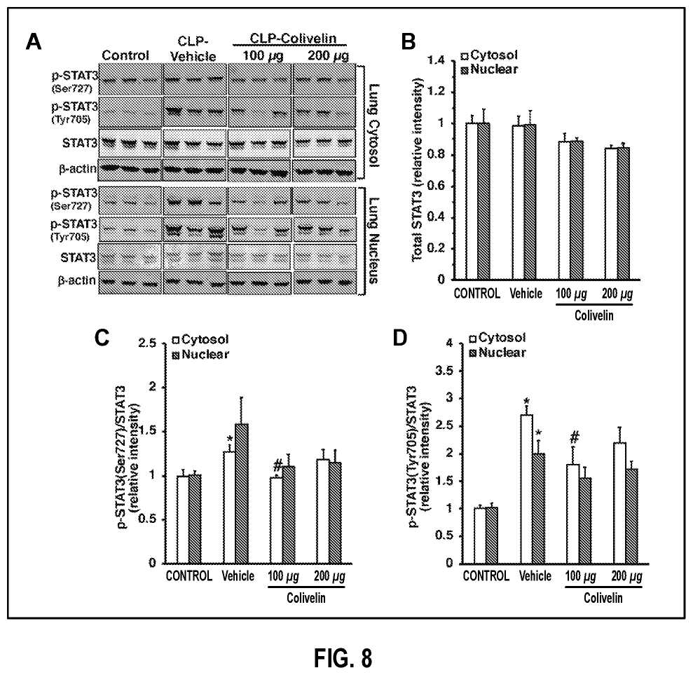

Since colivelin has been reported to activate STAT3 in vitro, Applicant next determined whether colivelin induced changes in STAT3 activation and intracellular localization in aortas and lungs. Control mice exhibited marginal levels of pSTAT3(Ser727), whereas the pSTAT3(Tyr705) was undetectable in both cytosol and nuclear compartments of thoracic aortas (FIG. 7). At 6 h after CLP, the levels of total STAT3 were reduced in the cytosol while they remained unchanged in the nucleus in vehicle-treated mice when compared to control mice. On the contrary, the expression of pSTAT3(Ser727) was significantly upregulated in the cytosol, while there was a trend towards increase in the nucleus; pSTAT3(Tyr705) was significantly upregulated in the cytosol and nuclear compartments when compared to basal levels of control mice, thus suggesting an overall activation of the transcription factor after sepsis. Interestingly, in thoracic aortas of colivelin-treated mice, cytosolic expression of both pSTAT3(Ser727) and pSTAT3(Tyr705) was significantly reduced. Colivelin treatment did not affect nuclear expression of pSTAT3(Ser727), while it inhibited pSTAT3(Tyr705) at the highest dose. Furthermore, the levels of total STAT3 were restored in the cytosol while they remained unchanged in the nucleus in colivelin-treated mice when compared to vehicle treatment (FIG. 7). In the lung, there was a constitutive expression of both pSTAT3(Ser727) and pSTAT3(Tyr705) in the cytosol and nuclear compartments of control mice (FIG. 8). At 6 h after CLP, the expression of pSTAT3(Ser727) was significantly upregulated in the cytosol, while there was a trend towards increase in the nucleus (P=0.063); pSTAT3(Tyr705) was significantly upregulated in the cytosol and nuclear compartments when compared to basal levels of control mice, thus suggesting an overall activation of the transcription factor also in the lung after sepsis. Interestingly, in the lung of colivelin-treated mice, cytosolic expression of both pSTAT3(Ser727) and pSTAT3(Tyr705) was significantly reduced when compared to vehicle-treatment. Nuclear expression of both pSTAT3(Ser727) and pSTAT3(Tyr705) was lower than vehicle-treated mice, but not statistically significant. In the lung, levels of total STAT3 were similar among the three groups of mice (FIG. 8).

Treatment with Colivelin Activates AMPK in Thoracic Aortas after CLP

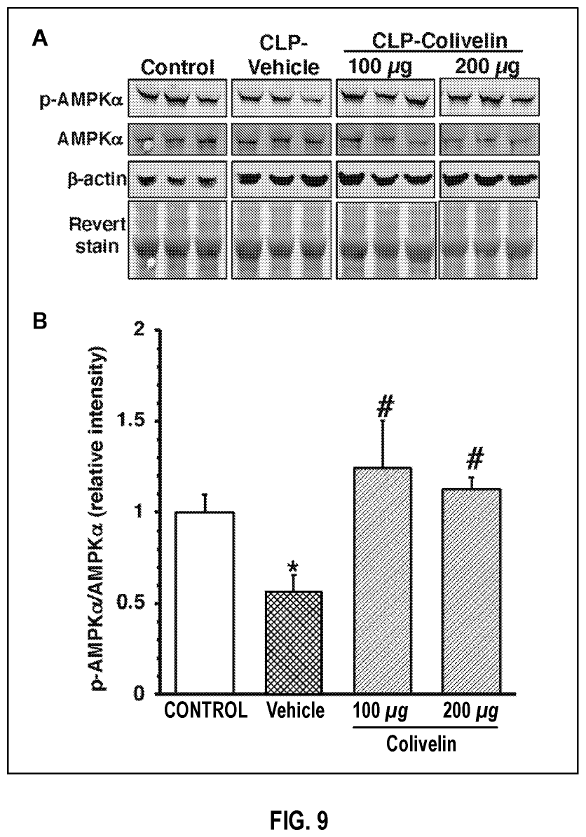

To further examine the molecular mechanism of colivelin, Applicant also determined the cytosolic activation of AMPK, the crucial regulator of mitochondrial control quality. At 6 h after CLP, the phosphorylated active pAMPKα1/α2 were reduced in the cytosol of thoracic aortas in vehicle-treated mice when compared to basal levels of control mice. Colivelin treatment significantly increased the ratio of the phosphorylated/total forms in a dose-independent manner, thus suggesting the restoration of the kinase function (FIG. 9).

Treatment with Colivelin Ameliorated Long-Term Outcomes after CLP

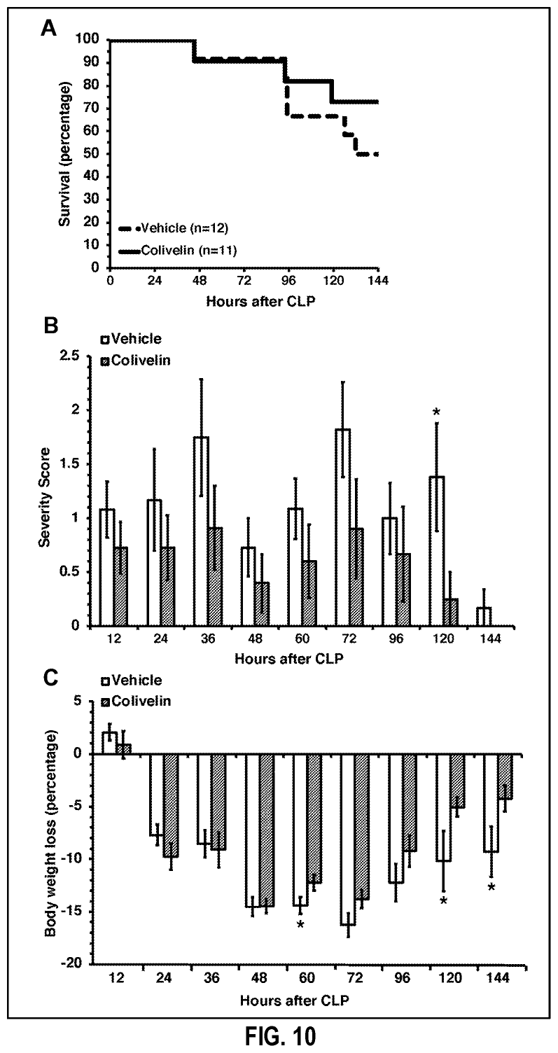

Given the early beneficial effects of colivelin on organ and endothelial injury induced by sepsis, Applicant sought to determine the effect of the peptide in long-term outcomes. In long-term studies, septic mice were treated with colivelin (100 μg/kg subcutaneously) or vehicle at 1 h, 3 h and 24 h after CLP and were monitored up to 7 days. To mimic the clinical condition, all mice also received antibiotic therapy for three days and fluid resuscitation for all the duration of the experimental period. The vehicle-treated group exhibited a survival rate of 50% as 6 out of 12 mice survived at 7 days after CLP. The colivelin-treated group experienced a slight, but not significant, increase of survival rate (72.6%) as 8 out of 11 mice survived at 7 days (FIG. 10A). Both vehicle- and colivelin-treated mice exhibited diarrhea, pilo-erection and signs of lethargy in the early 36 h after CLP. Symptoms declined at 48 h but increased again at later time after antibiotics discontinuation in both vehicle and colivelin-treated groups. However, colivelin-treated mice exhibited less severe signs of sepsis for all the duration of the observation period and survivor colivelin-treated mice were significantly healthier than survivor vehicle-treated mice at 6 and 7 days after CLP (FIG. 10B). Both vehicle- and colivelin-treated mice experienced a similar body weight loss in the first two days after CLP. However, at later time points vehicle-treated mice maintained a significant lower weight than colivelin-treated mice (FIG. 10C).

### Discussion

In the present work, Applicant demonstrated that increased plasma levels of biomarkers of endothelial permeability, inflammation and adhesiveness occurred at the early stage of experimental sepsis in mice (i.e., at 6 h after CLP) and coincided with structural changes of endothelial glycocalyx in the aorta and with inflammation of major organs. Applicant also demonstrated for the first time that colivelin, a potent synthetic humanin derivative, is a potential therapeutic compound to restore endothelial stability and improve outcomes of sepsis. Applicant found, in fact, that colivelin treatment attenuated infiltration of inflammatory cells in lung, kidney and liver, reduced the systemic release of the pro-inflammatory cytokines, and, when given as an adjunctive treatment to the standard fluid resuscitation and antibiotics, improved long-term recovery and health conditions of septic mice.

Being responsible for important physiological functions, such as hemostasis, vasomotor control, barrier integrity and immunological function, the endothelium is a critical cellular system for host survival following severe injury, including sepsis. Considering the systemic nature of sepsis, exposure to pathogen-associated molecular patterns and endogenous damage-associated molecular patterns may impair the structure and function of the endothelium and its glycocalyx layer. Several clinical studies have demonstrated elevated circulating levels of syndecan-1 as a marker of glycocalyx degradation in sepsis and are associated with organ dysfunction and mortality. Clinical studies have also profiled protein markers of endothelial activation in both the adult and pediatric populations and have reported significant associations with the severity of sepsis and septic shock, organ failure and mortality risks. Adhesion molecules, such as ICAM-1 and P-selectin, have been associated with poor outcomes of acute lung injury. In adult patients circulating P-selectin, measured at ICU admission, appears to be associated with sepsis development in time and it may have diagnostic value for sepsis when used with other endothelial markers. Angiopoietin-2, which is produced in endothelial cells and pre-stored in the Weibel-Palade bodies, has been correlated with sepsis severity and death and with acute kidney injury and respiratory failure. Other novel biomarkers have been proposed for the evaluation of endothelial dysfunction. Endoglin, or CD105, is a membrane-bound glycoprotein that serves as a co-receptor for members of the transforming growth factor-f and functions as an angiogenetic factor. Although not yet determined in sepsis, circulating levels of soluble endoglin have been shown to be higher in the serum of patients with cardiovascular diseases with a significant inflammatory component. Recent experimental and clinical studies have also supported a central role of PCSK9 in the clearance of pathogenic lipids such as the bacterial lipopolysaccharide (LPS) and in sepsis. Although mainly located in the liver, PCSK9 is also expressed in vascular smooth muscle and endothelial cells and its expression is increased by stimulation with LPS, suggesting a critical role of PCSK9 in vascular function. Plasma PCSK9 levels have been shown to serve as a late biomarker of the severity of illness in patients with severe trauma injury in ICU and sepsis and have been correlated to endothelial dysfunction in patients with chronic kidney disease.

Despite these data on association with poor prognosis, the pathophysiology of glycocalyx injury and endothelial dysfunction and its potential role as therapeutic targets in improving sepsis outcomes remain unclear. Applicant observed a distinct temporal profile of these circulating endothelial biomarkers and glycocalyx degradation. Applicant observed that plasma elevation of the glycocalyx component syndecan-1 occurred early after CLP procedure and correlated with an early elevation of plasma levels of the adhesion molecules ICAM-1 and P-selectin. This early increased expression of circulating syndecan-1 support the hypothesis that the shedding of the glycocalyx concomitantly occurs with the critical period of the inflammatory process of the endothelium and may precede angiogenetic events as angiopoietin-2 was elevated only at 18 h after CLP. Interestingly, Applicant has also identified other novel markers, such as endoglin and PCSK9.

Neutrophil infiltration is a crucial pathophysiological event of organ injury. In normal conditions, adhesion molecules responsible for leukocyte adhesion are embedded in the glycocalyx and are shielded from leukocytes rolling. Therefore, shedding of the glycocalyx allows for neutrophil infiltration. The early increase of syndecan-1 and adhesion molecules temporally correlated with neutrophil infiltration in lung, liver and kidney. In this regard, it is noteworthy that elevation of circulating syndecan-1 was associated with inflammatory biomarkers of neutrophil activation, including MPO, and was predictive of adverse clinical outcomes in patients with sepsis due to pneumonia.

To restore the endothelial permeability barrier and improve outcome in sepsis, Applicant tested the efficacy of colivelin, a new generation humanin peptide derivative (Chiba T et al. Development of a femtomolar-acting humanin derivative named colivelin by attaching activity-dependent neurotrophic factor to its n terminus: characterization of colivelin-mediated neuroprotection against alzheimer's disease-relevant insults in vitro and in vivo. J Neurosci (2005) 25:10252-61. doi:10.1523/JNEUROSCI.3348-05.2005). Humanin is a polypeptide containing 24 amino acids, which is encoded by short open reading frame in the mtDNA and acts as retrograde signaling molecule to regulate inflammation (Lionaki E et al. Differential protein distribution between the nucleus and mitochondria: Implications in aging. Front Genet (2016) 7:162. doi: 10.3389/fgene.2016.00162). Humanin was first identified in the cDNA associated with neuroprotective effects in Alzheimer's disease patients, and therefore recognized for its antiapoptotic properties (Hashimoto Y, Suzuki H, Aiso S, Niikura T, Nishimoto I, Matsuoka M. Involvement of tyrosine kinases and STAT3 in humanin-mediated neuroprotection. Life Sci (2005) 77:3092-104. doi: 10.1016/j.lfs.2005.03.031). Previous in vitro studies have demonstrated that humanin has cytoprotective effects in human aortic endothelial cells against oxidative stress. A synthetic analogue with enhanced potency, humanin-G, has also been reported to inhibit cell death in high-glucose-induced apoptosis in human umbilical vein endothelial cells. Another potent humanin derivative is colivelin, a hybrid peptide named composed of activity-dependent neurotrophic factor and fused at the C-terminus to a fragment of humanin (Chiba T, Yamada M, Hashimoto Y, Sato M, Sasabe J, Kita Y, et al. Development of a femtomolar-acting humanin derivative named colivelin by attaching activity-dependent neurotrophic factor to its n terminus: characterization of colivelin-mediated neuroprotection against alzheimer's disease-relevant insults in vitro and in vivo. J Neurosci (2005) 25:10252-61), which has been shown to provide beneficial effects in ischemia models in vivo (Zhao H, Feng Y, Wei C, Li Y, Ma H, Wang X, et al., Colivelin rescues ischemic neuron and axons involving JAK/STAT3 signaling pathway. Neuroscience (2019) 416:198-206. doi: 10.1016/j.neuroscience.2019.07.020). Here, it was demonstrated that in vivo treatment with colivelin reduced lung injury and reduced leukosequestration in lung, liver and kidney. These beneficial effects correlated with a significant reduction of circulating levels of syndecan-1, thus suggesting inhibition of glycocalyx shedding, when compared to vehicle treatment. To determine the beneficial effect of colivelin on glycocalyx and endothelium Applicant comprehensively assessed the vascular damage of thoracic aortas by transmission electron microscopy and found that colivelin treatment was associated with amelioration of glycocalyx structure, as evidenced by the presence of thick and complex bundles when compared to the loose and thin structure in mice receiving vehicle. Although analysis of the vascular wall was focused on glycocalyx structure, it was also found that vascular damage in vehicle-treated mice was characterized by the presence of damaged mitochondria in both endothelial and smooth muscle cells. Mitochondria in vascular smooth muscle and endothelial cells play a pivotal role in maintaining the structural integrity of the vascular wall, whereas their dysfunction leads to energy failure and contributes to inflammation via production of reactive oxygen species. Here, colivelin treatment also ameliorated mitochondrial structure. Thus, taken together, the data demonstrated that the peptide affords multifactorial beneficial effects against oxidative and metabolic stress and against neutrophil adhesiveness and activation at the vascular level. Many preclinical and clinical studies have demonstrated an association between inflammatory cytokines and glycocalyx degradation biomarkers. Here, colivelin treatment significantly blunted the systemic elevation of TNFα, MIP-1α, KC and IL-10, suggesting that the peptide interferes with the vicious cycle between impaired endothelial glycocalyx and neutrophil activation.

One of the most notable observations was that treatment with colivelin improved long-term wellbeing outcomes of mice subjected to sepsis. To mimic human sepsis management, mice were resuscitated with fluids and treated with antibiotics. It was observed that mice treated with a combination of fluids, antibiotics and colivelin experienced less severe clinical signs of sepsis up to 7 days after CLP when compared with animals treated only with fluids and antibiotics, suggesting beneficial effects of colivelin on recovery. The group of mice that received the adjunct therapy of colivelin also exhibited a higher, but not significant, survival rate (72.6%) than the group that received only vehicle in combinations with fluids and antibiotics (50%).

In evaluating the molecular mechanisms of colivelin, Applicant investigated the contribution of both STAT-3 and AMPK since these signaling pathways have been reported to be activated by humanin and its derivatives. STAT3 is a crucial transcription factor, which plays a role in development, inflammation, immunity, metabolism and cancer. In addition to its established role as a nuclear transcription factor, a pool of STAT3 has been described in the mitochondria. STAT3 in the mitochondria requires Ser727 but not Tyr705 phosphorylation and functions as a positive regulator of mitochondrial electron transport chain for ATP production. In vitro studies have shown that treatment with humanin and its analogues may exert protective functions through STAT3 phosphorylation. In a murine model of ischemic stroke, the beneficial effects on neuronal death and axonal remodeling of colivelin have also been associated with activation of STAT3 signaling. Previous studies have reported that expression of pSTAT3(Tyr705) increases in the lung, liver, and kidney in murine models of sepsis. However, these studies have not examined the subcellular localization of the different phosphorylated forms of STAT3. Applicant found that, in addition to the lung, pSTAT3(Tyr705) is also activated in the cytosol and nuclear compartments of the aorta at 6 h after CLP in vehicle-treated mice. Interestingly, distinct subcellular localization of the pSTAT3(Ser727) was also found, which increased in both aortas and lungs and was preferentially located in the cytosol. Non-canonical STAT3 activation through Ser727 phosphorylation has been recently demonstrated to serve as a crucial signaling intermediary for TLR4-induced glycolysis, macrophage metabolic reprogramming and inflammation. An intriguing finding of was that colivelin treatment inhibited the activation of STAT3 in the aorta and lung and was associated with improvement of endothelial damage and pulmonary protection. These data are in discrepancy with previous in vivo and in vitro studies demonstrating that colivelin may act as a potent activator of STAT3. A potential reason for this discrepancy on STAT3 activation by colivelin may be due to the different disease models. Applicant investigated the beneficial effects in an infection condition, while previous in vivo and in vitro studies have focused on conditions of neurodegeneration in Alzheimer's disease and ischemia and reperfusion injury models.

Applicant also investigated the contribution of AMPK signaling pathway. AMPK is a serine/threonine protein kinase, which is a regulator of energy metabolism and mitochondrial quality control. It was observed that the thoracic aorta of colivelin-treated mice had increased activation of AMPK. The literature suggests that humanin and humanin analogues may exert beneficial effects in oxidative stress by activation of AMPK. It has also been proposed that AMPK activation exerts anti-inflammatory effects in endotoxic shock in mice by inhibiting STAT3 signaling. Thus, it is plausible that the molecular mechanisms of the protective effect of colivelin in sepsis may be related to increased AMPK, which in turn inhibits phosphorylation of STAT3. Applicant's findings also support studies demonstrating that pharmacological activation of AMPK ameliorates organ injury in mice subjected to experimental sepsis.

### Materials and Methods

### Murine Model of Polymicrobial Sepsis

The investigation conformed to the National Institutes of Health Guide for the Care and Use of Laboratory Animals (Eighth Edition, 2011) and was approved by the Institutional Animal Care and Use Committee of the Cincinnati Children's Hospital Medical Center. Male C57BL/6 mice were obtained from Charles River Laboratories International, Inc. (Wilmington, MA). Mice were used at 3-5 months of age to mimic the equivalent for human ranges from 20-25 years (29). Male mice only were chosen for the experimentation to avoid interference from female hormonal fluctuations in sepsis responses during the estrous cycle. Mice were housed in pathogen-free conditions under a 10-h light/14-h dark cycle with free access to food and water. Mice were anesthetized with 2.0% isoflurane in 50% oxygen and polymicrobial sepsis was induced by cecal ligation and puncture (CLP) (30). After a midline laparotomy, the cecum was exteriorized, ligated and punctured twice with a 23-G needle. The cecum was then returned into the peritoneal cavity and the abdominal incision was closed. After the procedure, mice were randomly assigned to three treatment groups: a vehicle-treated group received distilled water (200 μl/mouse) intraperitoneally (i.p.); a 100 μg colivelin-treated group received the colivelin at 100 μg/kg i.p., and a 200 μg colivelin-treated group received the colivelin at 200 μg/kg i.p. at 1 h after CLP. The intraperitoneal injection was chosen to allow for a rapid uptake and bioavailability of the peptide. All groups of mice also received fluid resuscitation (35 ml/kg normal saline solution subcutaneously) immediately after, at 3 h and 12 h after the CLP procedure. To minimize pain at the surgical incision site, lidocaine hydrochloride (1%, 4 mg/kg total dose) was applied locally immediately after the procedure. Control mice did not undergo any surgical procedure; sham mice underwent laparotomy only without CLP. Mice were then sacrificed at 0, 6 and 18 h after CLP. Blood, lungs, kidneys, liver, and thoracic aortas were collected for biochemical assays.

### Long-Term Studies of Severity of Sepsis

In a separate study, another cohort of mice was subjected to the CLP procedure and was used for assessing health and moribundity conditions, and survival rate up to 7 days. Mice were divided into two treatment groups in a blind and random fashion: a vehicle-treated group received distilled water (200 μl/mouse), and a colivelin-treated group received colivelin (100 μg/kg subcutaneously) at 1 h, 3 h and 24 h after the CLP procedure. Twelve animals were included for each group. One animal was sacrificed because of unintentional extensive damage in the small intestine during surgical procedure and was excluded from the study. The subcutaneous injection was chosen to avoid further stress in the peritoneum since the animals also received intraperitoneal treatment of antibiotics. To mimic the clinical management of antimicrobial coverage, all mice received ceftriaxone (25 mg/kg) and metronidazole (12.5 mg/kg) intraperitoneally every 12 h after CLP for three days. To minimize pain, buprenorphine (0.05 mg/kg) was administered subcutaneously at 1 h after surgery and every 12 h for three days after surgery. All groups of mice also received fluid resuscitation (35 ml/kg normal saline with 5% dextrose subcutaneously) every 24 h for all the duration of the experimental period. Although some spontaneous death occurred given the acute severity of the disease, spontaneous death was not considered as endpoint for this study for ethical reasons. Animals were euthanized when they exhibited signs of moribundity. During the monitoring period a score system was developed according to the clinical signs of peritoneal sepsis (31, 32). Physical examination focused on four parameters: posture, feces consistency, eye appearance, hair coat. For each parameter a score 0 to 3 was given according to the abnormalities. Specifically, a score of 0 represents no symptoms; score of 1 represents minimum symptoms (awkward gait, loose stools, some watery ocular discharge, fuzzy facial fur); score of 2 represents mild symptoms (hunched or slow walk, watery stools, some yellow ocular discharge, rough hair coat); score of 3 represents severe symptoms (complete inability to move or lethargy, hemorrhagic diarrhea, red eyes with thick ocular discharge, pilo-erection). Weight loss of more than 20% was also considered a humane endpoint. Monitoring and weighing of the animals was performed daily by the laboratory personnel blinded to the treatment protocol and logged in a score sheet. Animals with cumulative scores >8 or weight loss >20% from the initial body weight were euthanized. Therefore, mice experiencing spontaneous death or euthanized within 7 days were defined as non-survivor mice. Animals that survived the entire observation period of 7 days were also euthanized and were defined as survivor mice.

### Myeloperoxidase Activity

Myeloperoxidase (MPO) activity was measured as an indicator of neutrophil infiltration in lung, kidneys and liver tissue. Tissues were homogenized in a solution containing 0.5% hexa-decyl-trimethyl-ammonium bromide dissolved in 10 mM potassium phosphate buffer (pH 7.0) and centrifuged for 30 min at 4000×g at 4° C. An aliquot of the supernatant was allowed to react with a solution of tetra-methyl-benzidine (1.6 mM) and hydrogen peroxide (0.1 mM). The rate of change in absorbance was measured by spectrophotometry at 650 nm. MPO activity was defined as the quantity of enzyme degrading 1 μmol of hydrogen peroxide/min at 37° C. and expressed in units per 100 mg weight of tissue.

### Histopathologic Analysis

Paraffin-embedded sections of thoracic aortas and lungs were stained with hematoxylin and eosin for morphological evaluation by three independent observers blinded to the treatment groups. Lung injury was also analyzed by a semiquantitative score based on the following histologic features: alveolar capillary congestion, infiltration of red blood cells and inflammatory cells into the airspace, alveolar wall thickness, and hyaline membrane formation (34). A score of 0 represented normal findings and scores of 1, 2, 3 and 4 represented minimal (<25% lung involvement), mild (25-50% lung involvement), significant (50-75% lung involvement) and severe (>75% lung involvement) injury, respectively. The four variables were summed to represent the lung injury score (total score, 0-16).

### Transmission Electron Microscopy

Glycocalyx structure was assessed by transmission electron microscopy (35). At 6 h after CLP, mice were again anesthetized with 2.0% isoflurane in 50% oxygen and perfused via cardiac puncture with a solution for lanthanum staining composed of 2% glutaraldehyde, 2% sucrose, 0.1 M sodium cacodylate buffer (pH 7.3), and 2% lanthanum nitrate. Thereafter, the aorta was harvested and diced in three to four pieces of approximately 1 mm³ each. Sections were immersed for 2 h in the lanthanum staining solution and then immersed overnight in a solution composed of 2% sucrose and 0.1 M sodium cacodylate buffer (pH 7.3). After washing in alkaline 2% sucrose and 0.03 M NaOH solution, sections were immersed in 2% osmium tetroxide and 2% lanthanum nitrate, embedded and cut with ultramicrotome. The sections were viewed and photographed on Hitachi H-7650 transmission electron microscope at 120 kV.

### Plasma Levels of Cytokines and Biomarkers of Endothelial Injury

Plasma levels of tumor necrosis factor-α (TNFα), interleukin (IL)-1β, IL-6, IL-10, keratinocytes-derived chemokine (KC), and macrophage inflammatory proteins (MIP-1α) were used as indices of the systemic inflammatory response and were evaluated by a commercially available multiplex array system (Milliplex, Millipore Corporation, Billerica, MA). Plasma levels of endoglin, intercellular adhesion molecule-1 (ICAM-1), P-selectin, proprotein convertase subtilisin/kexin type 9 (PCSK9), and angiopoietin-2 (Ang 2) were used as indices of endothelial injury and were evaluated by a commercially available multiplex array system (R&D Systems, Minneapolis, MN). Plasma levels of syndecan-1 were used as indices of glycocalyx damage and were evaluated by a mouse syndecan-1 sandwich-type enzyme-linked immunosorbent assay (ELISA) kit (Boster Biological Technology Co., California, US). Assays were performed using the protocols recommended by the manufacturer.

### Subcellular Fractionation

Subcellular fractionation was performed using a centrifugation model. Tissue samples of lung and thoracic aortas were homogenized in a buffer (50 mg tissue/100 μL) containing 0.32 M sucrose, 10 mM Tris-HCl (pH 7.4), 1 mM EGTA, 2 mM EDTA, 5 mM NaN3, 10 mM β-mercaptoethanol, 2 μM leupeptin, 0.15 μM pepstatin A, 0.2 mM phenylmethanesulfonyl fluoride, 50 mM NaF, 1 mM sodium orthovanadate and 0.4 nM microcystin. Samples were centrifuged at 1000×g for 10 min at 4° C. and the supernatants collected as cytosol extracts, which also contain membrane and mitochondria. The pellets were then solubilized in Triton buffer (1% Triton X-100, 250 mM NaCl, 50 mM Tris HCl at pH 7.5, 3 mM EGTA, 3 mM EDTA, 1 mM phenylme-thanesulfonyl fluoride, 0.1 mM sodium orthovanadate, 10% glycerol, 2 mM p-nitrophenyl phosphate, 0.5% NP-40 and 46 μM aprotinin). The lysates were rocked for 1 h and subsequently centrifuged at 15,000×g for 30 min at 4° C. and the supernatant collected as nuclear extracts. The Bradford protein assay was then used for quantitative determination of total proteins.

### Western Blot Analysis

Cytosol content of total AMPKα1/α2 and its phosphorylated form pAMPKα1/α2 (Santa Cruz Biotechnology, Dallas, TX, USA), cytosol and nuclear content of STAT3 and its phosphorylated forms pSTAT3(Tyr705) and pSTAT3(Ser727) (Cell Signaling Technology, Danvers, MA, USA) were determined by immunoblot analyses; β-actin was concomitantly probed with mouse anti-β-actin (Santa Cruz Biotechnology) as a loading control for both cytosol and nuclear proteins. Extracts were heated at 70° C. in equal volumes of 4× Protein Sample Loading Buffer. Twenty-five μg of proteins were loaded per lane on a 10% Bis-Tris gel. Proteins were separated electrophoretically and transferred to nitro-cellulose membranes. The immunoreaction was detected by near-infrared fluorescence. Membranes were blocked with Odyssey blocking buffer (LI-COR Biotechnology, Lincoln, NE, USA) and incubated with primary antibodies. Membranes were washed in PBS with 0.1% TWEEN 20 and incubated with near infrared fluorescent dye-conjugated secondary antibodies (IRDye goat anti-rabbit and anti-mouse IgG; LI-COR Biotechnology). Immunoblotting was performed by using the IBind Flex Western System (Thermo Fischer Scientific, Waltham, MA, USA) that uses sequential lateral flow to perform blocking and antibody binding. The Odyssey LI-COR scanner (LI-COR Biotechnology) was used for detection. Fold changes of relative intensity of proteins were calculated versus mean value of control mice upon data normalization with 0-actin by NIH ImageJ 1.53 k software (36). Normalization and quantification for AMPKα1/α2 was also validated by Revert total protein stain and Empiria Studio analysis (LI-COR Biotechnology).

### Materials

Unless otherwise stated, all chemicals were obtained from Sigma-Aldrich (St. Louis, MO).

### Statistical Analysis

Statistical analysis was performed using SigmaPlot 14.0 (Systat Software, San Jose, CA, USA). Data in figures and text are expressed means±SEM or median with 25th and 75th percentile of n observations (n=3-17 animals for each group). The results were examined by analysis of variance followed by the Student-Newman-Keuls correction post hoc t-test. Statistical analysis of damage scores was performed using the non-parametric Mann-Whitney test. The Gehan-Breslow test was used to compare differences in survival rates (n=11-12 animals for each group). A value of P<0.05 was considered significant.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A method of treating sepsis in an individual in need thereof, comprising administering a therapeutically effective amount of a humanin protein, or an analog thereof, to said individual.

2. The method of claim 1, wherein said sepsis is accompanied by one or both of sepsis-associated endothelial dysfunction and organ injury.

3. The method of claim 1, wherein said humanin protein or analog thereof has at least 90%, or at least 95% sequence homology to a peptide having a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8.

4. The method of claim 1, wherein said humanin protein or analog thereof is selected from HN, HNG, HNA, AGA-HNG, HN17, HNG17, AGA-C8R-HNG17, colivelin, and combinations thereof.

5. The method of claim 1, wherein said humanin protein or analog thereof is colivelin (SEQ ID NO: 8).

6. The method of claim 1, wherein said humanin protein or analog thereof is PEGylated.

7. The method of claim 1, wherein said humanin protein or analog thereof is administered as a pro-drug.

8. (canceled)

9. The method of claim 1, wherein said administering results in improvement of lung injury, reduced leukosequestration in lung, liver and kidney, a reduction of circulating syndecan-1, and combinations thereof.

10-16. (canceled)

17. The method of claim 1, wherein said humanin protein or analog thereof is administered for at a period of at least one hour, or at least one day, or at least two days, or at least three days, or at least four days, or at least five days, or at least six days, or at least one week.

18. The method of claim 1, wherein said humanin protein or analog thereof is administered within two hours of admission or diagnosis, within three hours of admission or diagnosis, within four hours of admission or diagnosis, within five hours of admission or diagnosis, or within six hours of admission or diagnosis.

19. The method of claim 1, wherein said humanin protein or analog thereof is administered in an amount of from about 50 μg/kg to 1000 μg/kg, from about 100 μg/kg to about 800 μg/kg, from about 200 μg/kg to about 600 μg/kg, or from about 300 μg/kg to about 500 μg/kg.

20. The method of claim 1, wherein said sepsis is due to a bacteria selected from Staphylococcus aureus, Pseudomonas aeuroginosa, Klebsiella species, Streptococcus species, Salmonella species, Shigella species, Mycobacterium tuberculosis, Enterococcus species, Enterobacteriaceae, E. coli, Clostridium species, Neisseria gonnorrhoea, Acinetoebacter baumannii, Campylobacter species, and combinations thereof.

21. The method of claim 1, wherein said humanin protein or analog thereof is co-administered with one or both of a fluids and an antibiotic.

22. The method of claim 21 wherein said antibiotic is a broad-spectrum antibiotic.

23-25. (canceled)

26. A method of treating an individual for sepsis, comprising
a. detecting an elevated level of a biomarker selected from tumor necrosis factor-α (TNFα), interleukin (IL)-1β, IL-6, IL-10, keratinocytes-derived chemokine (KC), macrophage inflammatory proteins (MIP-1α), endoglin, PCSK9, ICAM-1, P-selectin, syndecan-1, and combinations thereof in said individual; and
b. administering a therapeutically effective amount of a humanin protein, or analog thereof, to said individual;
wherein said humanin protein, or analog thereof, is optionally provided in a pharmaceutically acceptable carrier.

27. The method of claim 26, wherein said humanin protein or analog thereof has at least 90%, or at least 95% sequence homology to a peptide having a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8.

28. The method of claim 26, wherein said humanin protein or analog thereof is selected from HN, HNG, HNA, AGA-HNG, HN17, HNG17, AGA-C8R-HNG17, colivelin, and combinations thereof.

29. (canceled)

30. The method of claim 26, further comprising administering one or both of an antibiotic or fluid to said individual.

31. The method of claim 26, wherein said humanin protein or analog thereof is PEGylated.

32. The method of claim 26, wherein said humanin protein or analog thereof is administered as a pro-drug.

33-40. (canceled)


SEQUENCE LISTING (11 sequences)

SEQ ID NO: 1 - protein, 23 aa, Homo sapiens
MAPRGFSCLLLTSEIDLPVKRRA

SEQ ID NO: 2 - protein, 24 aa, synthetic construct
MAPRGFSCLLLLTGEIDLPVKRRA

SEQ ID NO: 3 - protein, 24 aa, synthetic construct
MAPRGFSALLLLTSEIDLPVKRRA

SEQ ID NO: 4 - protein, 24 aa, synthetic construct
MAAGAFSCLLLLTSEIDLPVKRRA

SEQ ID NO: 5 - protein, 17 aa, synthetic construct
PRGFSCLLLLTSEIDLP

SEQ ID NO: 6 - protein, 17 aa, synthetic construct
PRGFSCLLLLTGEIDLP

SEQ ID NO: 7 - protein, 17 aa, synthetic construct
PAGASRLLLLTGEIDLP

SEQ ID NO: 8 - protein, 26 aa, synthetic construct
SALLRSIPAPAGASRLLLLTGEIDLP

SEQ ID NO: 9 - protein, 11 aa, synthetic construct
CLLLTSEIDLP

SEQ ID NO: 10 - protein, 5 aa, synthetic construct
LLLLT

SEQ ID NO: 11 - protein, 5 aa, synthetic construct
EIDLP